(19) United States
(12) Patent Application Publication
(10) Pub. No.: US 2007/0282179 A1
(43) Pub. Date: Dec. 6, 2007

(54) METHOD AND APPARATUS FOR ASSESSING THE MOLECULAR WATER BINDING OF DEEP TISSUE IN VIVO USING NONIONIZING RADIATION

(75) Inventors: Sean Merritt, Lake Forest, CA (US); Bruce J. Tromberg, Irvine, CA (US); Albert E. Cerussi, Lake Forest, CA (US); Anthony J. Durkin, Irvine, CA (US); So Hyun Chung, Irvine, CA (US)

Correspondence Address: MYERS DAWES ANDRAS & SHERMAN, LLP, 19900 MACARTHUR BLVD.,, SUITE 1150, IRVINE, CA, 92612, US

(73) Assignee: The Regents of the University of California, Oakland, CA (US)

(21) Appl. No.: 11/757,673

(22) Filed: Jun. 4, 2007

Related U.S. Application Data
(60) Provisional application No. 60/811,225, filed on Jun. 5, 2006.

(51) Int. Cl. A61B 5/00

(52) U.S. Cl. 600/315

(57) ABSTRACT

A method of optically analyzing tissue in vivo in an individual to obtain a unique spectrum for the tissue of the individual includes the steps of optically measuring the tissue of the individual using broadband diffuse optical spectroscopy (DOS) to measure a normalized tissue water spectrum of the individual or noninvasively optically line scanning a tissue site on the individual at a plurality of points, then determining spectral differences between the normalized tissue water spectrum and a pure water spectrum at each point of a line scan, generating a bound water index (BWI) corresponding to the spectral differences, and identifying a tissue state corresponding to the scanned tissue based on the BWI.

### RELATED APPLICATIONS

The present application is related to U.S. Provisional Patent Application Ser. No. 60/811,225, filed on Jun. 5, 2006, which is incorporated herein by reference and to which priority is claimed pursuant to 35 USC 119.

### GOVERNMENT RIGHTS

This invention was made with Government Support under Grant No. RR001192, awarded by the National Institutes of Health. The Government has certain rights in this invention.

### BACKGROUND OF THE INVENTION

1. Field of the Invention

The invention relates to the field of medical optical spectroscopy and in particular to a method and apparatus for assessing bond water in deep tissues in vivo using nonionizing radiation.

2. Description of the Prior Art

Water is an important chromophore which can mark physiological and pathological changes of tissues. Several optical research groups have recognized the importance of water and measured increased water fraction in tumor tissues compared to normal tissues. Water concentration has been shown to have a correlation even with cancer progress and size of tumors. Water in tissues is also being measured routinely by MRI groups by observing intensity of pixels in T2 weighted images. In T2 weighted images, higher intensity has been observed in necrotic regions than cellular regions.

In spite of these efforts to measure tissue water concentrations, the water fraction itself may not enough because there are different states of water in tissues. Measurement of these different states can give more detail about pathological changes of tissues. For example, cellularity, which is the state of a tissue or other mass with regard to the degree, quality, or condition of cells present in it, is related to amount of protein bound water which is different from free water. MRI research groups have measured mobility of diffusing water using diffuse weighted MRI in order to measure the states of water, and they found different apparent diffuse coefficients of water (ADCw) in tumor and normal tissues. By comparing ADCw and histological data, it is shown that ADCw has a negative correlation with cellularity. However, because of heterogeneity of cell distribution in tumor tissues and various characteristics of different kind of tumor tissue, it is hard to get identical trends or values of ADCw from tumor tissue.

### BRIEF SUMMARY OF THE INVENTION

In the illustrated embodiment, we measured water state differences in malignant tumor tissues and normal tissues using broadband diffuse optical spectroscopy (DOS). DOS can measure absorption and scattering separately at few centimeters depth in a large volume of tissues using photon migration theory. The broadband DOS is combination of measurement in the modulated frequency domain mode and steady state spectroscopy mode, providing spectra and volume fractions of many tissue chromophores in near-infrared range. DOS can measure tissue physiological properties non-invasively (no compression) without high cost and ionizing radiation risk. These characteristics allows DOS to be easily used in clinical studies.

By using a high resolution spectrometer in a DOS system, we could observe a shift of broadened water peak around 980 nm in tissue. This is considered due to more binding of water molecules to macromolecules such as protein. The binding of water to macromolecules decreases water molecule's vibration level by restricting mobility of water molecules. The pathological changes of tumor tissues, including cellularity changes and necrosis increases, should be related to water state changes as well. This can be measured by calculating differences between a pure water spectrum and a tumor or tissue water spectrum for the subject. The differences can be represented with an index called bound water index (BWI). Absolute values of spectral differences between normalized tissue water spectra and pure water spectra were summed and divided by the number of points in sum to form BWI.

More particularly, the illustrated embodiment of the invention is directed to a method of optically analyzing tissue in vivo in an individual to obtain a unique spectrum for the tissue of the individual including the steps of noninvasively optically line scanning a tissue site on the individual at a plurality of points using broadband diffuse optical spectroscopy (DOS) to measure a normalized tissue water spectrum of the individual, determining spectral differences between the normalized tissue water spectrum and a pure water spectrum at each point of a line scan, generating a bound water index (BWI) corresponding to the spectral differences, and identifying a tissue state corresponding to the scanned tissue based on the BWI. In the illustrated embodiment the BWI is generated by summing an absolute value of the spectral differences at the plurality of points of the line scan to obtain a sum, and dividing by the number of points in sum to form the bound water index (BWI).

In one embodiment the step of determining spectral differences between the normalized tissue water spectrum and a pure water spectrum at each point of the line scan includes determining absorption spectral differences at each point of the line scan.

In another embodiment the step of determining absorption spectral differences at each point of the line scan includes determining absolute absorption spectral differences.

In the illustrated embodiment the step of determining absorption spectral differences comprise determining absorption spectral differences in the 650-1000 nm wavelength range. The step of identifying a tissue state corresponding to the scanned tissue based on the BWI includes distinguishing between malignant and normal tissues. The step of distinguishing between malignant and normal tissues includes distinguishing between malignant and normal tissues in breast tissue.

In an embodiment the step of determining spectral differences between the normalized tissue water spectrum and a pure water spectrum at each point of a line scan further includes temperature compensating the determined spectral differences.

In yet another embodiment the step determining spectral differences between the normalized tissue water spectrum and a pure water spectrum at each point of a line scan includes subtracting out spectra of oxy- and deoxy-hemoglobin and lipid from a measured absorption spectrum to obtain only a water spectrum.

In one embodiment the step of subtracting out spectra of oxy- and deoxy-hemoglobin and lipid from a measured absorption spectrum to obtain only a water includes subtracting out spectra of oxy- and deoxy-hemoglobin and lipid from a measured absorption spectrum in the wavelength range of 935 to 998 nm.

The invention further includes an apparatus for performing each of the methods described above.

The invention is further directed to a program or set of instructions recorded on a medium for controlling an apparatus to perform each of the methods described above.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a spectral graph of optical absorption of normalized water spectra in range of 935 to 998 nm of pure water at 37° C., normal breast tissue and tumor tissue.

FIG. 2 is a graph of Tissue Optical index (TOI) on top and of Bound Water Index (BWI) on the bottom as a function of position in line scanned breast tissues from one of the subjects. This subject had a tumor from approximately positions −3 to 3.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

Differences in tissue water state have been measured in normal and malignant breast tissues. Broadband diffuse optical spectroscopy (DOS) has been used to acquire 650-1000 nm absorption spectra of normal and tumor breast tissues from 7 patients in vivo. The absolute values of spectral differences between normalized tissue water spectra and pure water spectra were summed and divided by the number of points in sum to form the bound water index (BWI).

In all subjects in the study of the illustrated embodiment, the average BWIs of all line scan points were significantly lower in tumor tissues (1.62±0.27×10⁻³) than normal tissues (3.06±0.51×10⁻³, Wilcoxon Ranked Sum Test z=0.003 and power=0.98). These results imply that water in tumors behaves more like free water than the water in normal tissues. The BWI is therefore a functional parameter that distinguishes between malignant and normal tissues in the breast. Although breast tissue is the tissue of study in the illustrated embodiment, it is to be explicitly understood that any human or animal tissue may be subjected to the method and apparatus of the invention.

Bound water also has been measured by MRI groups by measuring diffusion of water in tissues. In diffusion-weighted MRI, the ADC (apparent diffusion coefficient) of water has been shown to be associated with the fraction changes of intracellular and extracellular water. This cellular level water fraction changes observed by diffusion weighted MRI can be compared to the BWI measured by DOS. The actual bound water fraction of tissues is currently unknown and will be figured out.

Methods

Instrumentation

The instrumentation details of a broadband DOS system have been described in literature and hence can be taken as known, e.g. Cerussi et al. JBO 11(4) 044005, 2006; and Bevilacqua et al. Appl. Opt. 39, 6498-6507, 2000. See also the instrument described in patent application Ser. No. 10/191,693, which is incorporated herein by reference. The core characteristic of the broadband DOS system insofar as the illustrated embodiment is concerned is the combination of modulated multi frequency domain (FDPM component) and broadband steady state domain (SS component). For the FDPM part, the lights from 658, 682, 785, 810, 830 and 850 nm laser diodes were amplitude modulated from 50 to 600 MHz sweeping 401 frequencies by combining a DC current and RF modulation current provided by a network analyzer. Such values are exemplary only and many other values can be chosen consistent with the teachings of the present invention. The laser diodes delivered less than 20 mW optical power to the tissue. An avalanche photodiode detector (APD) detected phase and amplitude of the diffused optical signals after the light's propagating through the tissue. The detected phases and amplitudes were compared to those of the source by the network analyzer which worked as a fast electronic heterodyning digitizer. The SS system is composed of a high intensity tungsten-halogen light source and a high resolution spectrometer (B&W Tek 611).

Because bound water calculation algorithm is based on the absolute wavelengths of the spectrum, automatic and stable calibration of the spectrometer is necessary. The SS system enabled acquisition of continuous absorption spectrum even in the water spectrum wavelength range (>935 nm) longer than the wavelengths of the laser diodes.

We employed a conventional handheld probe as described in the paper of Cerussi et al. above to measure breast cancer patients. In the handheld probe, optical fibers for the source of FDPM system, for the source light of SS system and for the detector of SS system are secured. The APD is housed in the handheld probe directly. The distance between sources and detectors of FDPM and SS systems can be changed by moving a plastic attachment on the probe.

To remove artifacts of cable length and source strength variability of FDPM system, a tissue-simulating phantom with known optical property has been used as described in the paper of Cerussi et al. above. For the SS system, an integrating sphere has been used to eliminate wavelength dependent artifact of the system.

Spectral Processing

Reduced scattering coefficient (μₛ) and absorption coefficient (μₐ) have been measured and separated by FDPM theory and practice. The details about this theory and practice have been well described in the literatures, e.g. the two papers cited above and Pham et al., Rev. Sci. Instr., 71, 2500, 2000. The volume fraction of major chromophores (ctHHb, ctHbO₂, ctH₂O and lipid concentration) have been calculated as described in Cerussi et al. above, but there were a few differences in the employed molar extinction values. For water, the molar extinction coefficients were obtained by our group by measuring distilled water in a spectrophotometer in various temperatures. Those water spectra at various temperatures have been used in the post-processing step to cancel out temperature effect. The employed molar extinction values of lipid were obtained from conventional values published in the literature by van Veen et al. “Determination of VIS-NIR absorption coefficients of mammalian fat, with time- and spatially resolved diffuse reflectance and transmission spectroscopy,” OSA Annual BIOMED Topical Meeting, 2004.

Post-Processing for Bound Water Measurements

The obtained μₐ values were post processed to measure different states of water. In order to get only water spectrum, spectra of oxy- and deoxy-hemoglobin and lipid were subtracted from the original absorption spectrum under assumption that only oxy- and deoxy-hemoglobin, lipid and water are major influential chromophores in breast tissues. Then the obtained water spectrum was compared to a pure water spectrum of breast temperature. The difference between the tissue water spectrum and the pure water spectrum was calculated by subtracting pure water spectra from normalized tissue water spectra in the wavelength range from 935 nm to 998 nm. Then, the absolute values of the differences were summed and divided by number of points in sum to form bound water index (BWI).

In-Vivo Breast Measurements

Line scans were performed to measure in-vivo malignant breast tumors. There were seven subjects (age: 48.3±7.4) and all of them were measured by the same spectrometer (B&W Tek 611) which performed auto calibration. The methods used for characterizing the optical and physiological properties of line scans were the same as the paper of Cerussi et al. above. The same arbitrary DOS parameters were employed to characterize physiological properties of tumor and normal tissues. In order to test statistical significance, two-tailed Wilcoxon/Kruskal-Wallis Rank Sums test was employed.

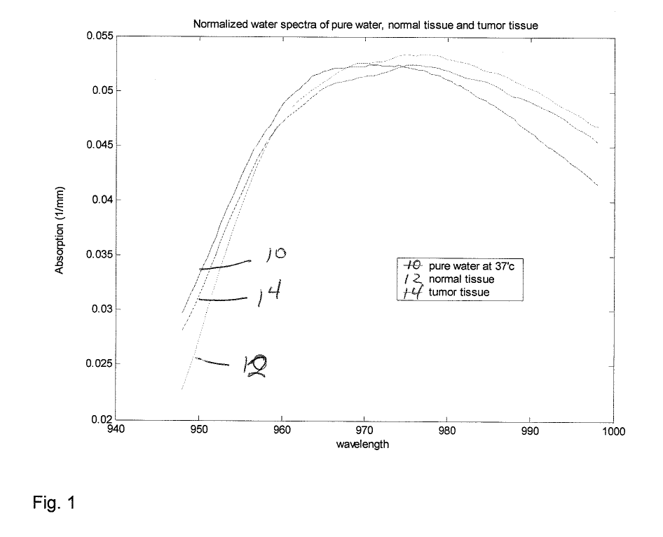

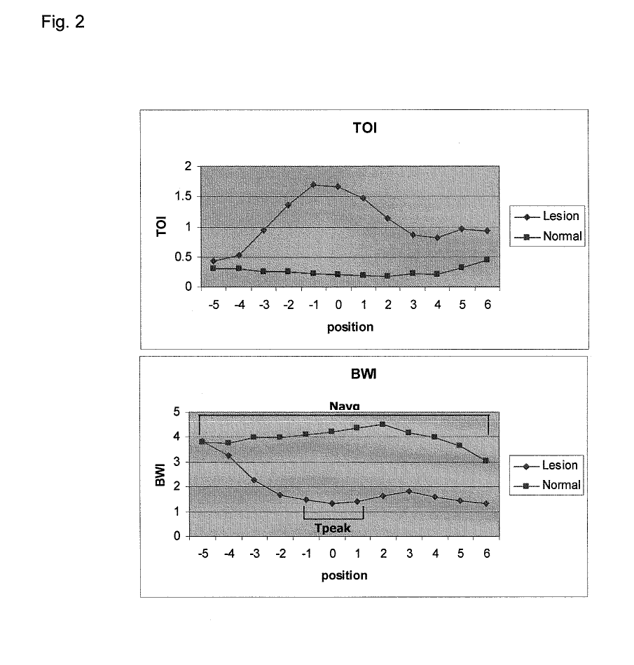

Tissue absorption spectra were acquired from the seven subjects who had malignant breast cancers. The measurements were performed contralaterally so that spectra could be acquired from both normal tissues and tumor tissues from the same subject. In FIG. 1, we can observe water peak changes in normal and tumor tissues in graphs 12 and 14 respectively as compared to pure water peak after normalization in graph 10. As explained above in the section, “methods”, BWI was acquired by calculating the differences between the pure water spectra and the tissue water spectra. BWIs calculated on every line scan point were compared to tissue optical index (TOI: ctH2O×ctHHb/Lipid) and ultra sound reports of each patient. BWIs were lower in tumor area than normal tissue area in all subjects as depicted in the graph of FIG. 2 which is contrary to the trend of TOI. Two arbitrary parameters have been employed to compare BWI of normal tissues and tumor tissues. In normal tissues, BWIs of all line scan points were averaged to be represented as Navg. In tumor tissues, the minimum three points of BWI were averaged to form Tpeak. These parameters were acquired from seven patients and the values are shown on Table 1. To test statistical significance of this result, two-tailed Wilcoxon/Kruskal-Wallis Rank Sums test has been performed between average values of all line scan points of normal and tumor tissues and we got z-value of 0.003 with power 0.98, which means the BWIs of normal and tumor data sets were significantly different from each other.

TABLE 1
BWIs of Navg (normal tissues) and Tpeak (Tumor tissues)
from 7 subjects

| Avg. ± Std. (n = 7) | Navg | Tpeak |
|---|---|---|
| BWI | 3.06 ± 0.51 | 1.76 ± 0.30 |

In summary, in the illustrated embodiment tissue bound water has been measured in breast cancer tissues using broadband DOS. BWI of normal tissues and malignant tumor tissues were different with statistical significance. Lower BWI in tumor tissues means that there is more free water in tumor tissues than normal tissues. We hypothesize that this is due to edema of tumor tissues. We contend that there is a statistical significance of the BWI in normal and cancer tissues. Furthermore, water states in a benign tumor and cyst as measured differentiate the water state in a malignant tumor from that of other types of lesion. In order to test the accuracy of bound water measurement and to acquire fraction of bound water, gelatin phantoms can be measured by broadband DOS and nuclear magnetic resonance both and the data can be compared. Also, the BWI can be compared to the apparent diffusion coefficient of water measured in vivo. Therefore tissue water states in cancer tissues under chemotherapy can be measured to show that BWI is helpful to monitor early response to chemotherapy agents for breast and other types of cancer patients.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A method of optically analyzing tissue in vivo in an individual to obtain a unique spectrum for the tissue of the individual comprising:
noninvasively optically line scanning a tissue site on the individual at a plurality of points using broadband diffuse optical spectroscopy (DOS) to measure a normalized tissue water spectrum of the individual;
determining spectral differences between the normalized tissue water spectrum and a pure water spectrum at each point of a line scan;
summing an absolute value of the spectral differences at the plurality of points of the line scan to obtain a sum;
dividing by the number of points in sum to form a bound water index (BWI); and
identifying a tissue state corresponding to the scanned tissue based on the BWI.

2. The method of claim 1 where determining spectral differences between the normalized tissue water spectrum and a pure water spectrum at each point of the line scan comprises determining absorption spectral differences at each point of the line scan.

3. The method of claim 1 where determining absorption spectral differences at each point of the line scan comprises determining absolute absorption spectral differences.

4. The method of claim 2 where determining absorption spectral differences comprise determining absorption spectral differences in the 650-1000 nm wavelength range.

5. The method of claim 1 where identifying a tissue state corresponding to the scanned tissue based on the BWI comprises distinguishing between malignant and normal tissues.

6. The method of claim 5 where distinguishing between malignant and normal tissues comprises distinguishing between malignant and normal tissues in breast tissue.

7. The method of claim 1 where determining spectral differences between the normalized tissue water spectrum and a pure water spectrum at each point of a line scan further comprises temperature compensating the determined spectral differences.

8. The method of claim 1 where determining spectral differences between the normalized tissue water spectrum and a pure water spectrum at each point of a line scan comprises subtracting out spectra of oxy- and deoxy-hemoglobin and lipid from a measured absorption spectrum to obtain only a water spectrum.

9. The method of claim 7 where determining spectral differences between the normalized tissue water spectrum and a pure water spectrum at each point of a line scan comprises subtracting out spectra of oxy- and deoxy-hemoglobin and lipid from a measured absorption spectrum to obtain only a water spectrum.

10. The method of claim 8 where subtracting out spectra of oxy- and deoxy-hemoglobin and lipid from a measured absorption spectrum to obtain only a water comprises subtracting out spectra of oxy- and deoxy-hemoglobin and lipid from a measured absorption spectrum in the wavelength range of 935 to 998 nm.

11. An apparatus for optically analyzing tissue in vivo in an individual to obtain a unique spectrum for the tissue of the individual comprising:
means for noninvasively optically line scanning a tissue site on the individual at a plurality of points using broadband diffuse optical spectroscopy (DOS) to measure a normalized tissue water spectrum of the individual;
means for determining spectral differences between the normalized tissue water spectrum and a pure water spectrum at each point of a line scan;
means for summing an absolute value of the spectral differences at the plurality of points of the line scan to obtain a sum;
means for dividing by the number of points in sum to form a bound water index (BWI); and
means for identifying a tissue state corresponding to the scanned tissue based on the BWI.

12. The apparatus of claim 11 where the means for determining spectral differences between the normalized tissue water spectrum and a pure water spectrum at each point of the line scan comprises means for determining absorption spectral differences at each point of the line scan.

13. The apparatus of claim 11 where the means for determining absorption spectral differences at each point of the line scan comprises means for determining absolute absorption spectral differences.

14. The apparatus of claim 12 where the means for determining absorption spectral differences comprise means for determining absorption spectral differences in the 650-1000 nm wavelength range.

15. The apparatus of claim 11 where the means for identifying a tissue state corresponding to the scanned tissue based on the BWI comprises means for distinguishing between malignant and normal tissues.

16. The apparatus of claim 15 where the means for distinguishing between malignant and normal tissues comprises means for distinguishing between malignant and normal tissues in breast tissue.

17. The apparatus of claim 11 where the means for determining spectral differences between the normalized tissue water spectrum and a pure water spectrum at each point of a line scan further comprises means for temperature compensating the determined spectral differences.

18. The apparatus of claim 11 where the means for determining spectral differences between the normalized tissue water spectrum and a pure water spectrum at each point of a line scan comprises means for subtracting out spectra of oxy- and deoxy-hemoglobin and lipid from a measured absorption spectrum to obtain only a water spectrum.

19. The apparatus of claim 17 where the means for determining spectral differences between the normalized tissue water spectrum and a pure water spectrum at each point of a line scan comprises means for subtracting out spectra of oxy- and deoxy-hemoglobin and lipid from a measured absorption spectrum to obtain only a water spectrum.

20. The apparatus of claim 18 where the means for subtracting out spectra of oxy- and deoxy-hemoglobin and lipid from a measured absorption spectrum to obtain only a water comprises means for subtracting out spectra of oxy- and deoxy-hemoglobin and lipid from a measured absorption spectrum in the wavelength range of 935 to 998 nm.

21. A method of optically analyzing tissue in vivo in an individual to obtain a unique spectrum for the tissue of the individual comprising:
noninvasively optically line scanning a tissue site on the individual at a plurality of points using broadband diffuse optical spectroscopy (DOS) to measure a normalized tissue water spectrum of the individual;
determining spectral differences between the normalized tissue water spectrum and a pure water spectrum at each point of a line scan;
generating a bound water index (BWI) corresponding to the spectral differences between the normalized tissue water spectrum and a pure water spectrum at each point of a line scan; and
identifying a tissue state corresponding to the scanned tissue based on the BWI.
